(19) United States
(12) Patent Application Publication
(10) Pub. No.: US 2004/0260169 A1
(43) Pub. Date: Dec. 23, 2004

(54) Nonlinear noise reduction for magnetocardiograms using wavelet transforms

(76) Inventor: Karsten Sternnickel, Albany, NY (US)

Correspondence Address: KATTEN MUCHIN ZAVIS ROSENMAN, 575 MADISON AVENUE, NEW YORK, NY, 10022-2585, US

(21) Appl. No.: 10/487,513

(22) PCT Filed: Sep. 20, 2002
(86) PCT No.: PCT/US02/29920

(30) Foreign Application Priority Data
Sep. 21, 2001 (US) 60324009

(51) Int. Cl. A61B 5/05; A61B 5/04

(52) U.S. Cl. 600/409; 600/509

(57) ABSTRACT

A two-part method for reducing the noise contribution in a composite signal using the wavelet transform is described. The procedure involves the identification of subspaces in the reconstructed state space created by dynamical processes (either by deterministic noise or the signal itself), the separation of different subspaces and the separation of subspaces from stochastic noise. The method is used for non-linear de-noising (NLD) of magnetocardiograph or electrocardiograph time series signals by performing local projections in the reconstructed state space using the wavelet transform to identify and describe deterministic structures. Subspaces generated by any deterministic process are located and separated independently of its source.

### BACKGROUND OF THE INVENTION

1. Field of Invention

The present invention relates generally to the field of magnetocardiography and electrocardiography. More specifically, the present invention is related to nonlinear noise reduction for magnetocardiograms using wavelet transforms.

2. Discussion of Prior Art

Magnetocardiography (MCG) is the measurement of magnetic fields emitted by the heart from small currents by electrically active cells of the heart muscle. It is a noninvasive diagnostic method still not introduced into routine clinical practice.

Magnetocardiography consists of measurements of time-varying magnetic fields generated above the torso (or maternal abdomen in fetal magnetocardiography) by the electrophysiological processes in the heart. The measurement of these fields over the torso provides information which is complementary to that obtained by electrocardiography, and is used especially in diagnosing abnormalities of heart function.

Due to the extremely weak strength of these signals (one millionth or less of the earth's magnetic field), currently only Superconducting QUantum Interference Devices (SQUIDs) are capable of such a task. Such sensors operate only at very low (cryogenic) temperatures and must be placed inside a special enclosure (cryostat). Its external walls are at room temperature, while inside the low temperature is attained, usually by filling by a low-temperature liquid (cryogen), most typically liquid helium. The cryostat with SQUID sensors is positioned close to, but without any contact with the human body. It is possible, in principle, to replace liquid helium by liquid nitrogen, by utilizing high temperature superconductor SQUIDs (HTSQUIDs) and gradiometers. Although this would greatly simplify the handling, to date demonstrated HTS-based MCG systems have not been entirely practical.

Although magnetocardiography has several advantages compared with electrocardiography, a breakthrough for a practical clinical use is still missing. Therefore, it is necessary to develop convincing and attractive results for medical doctors, and to reduce the costs of SQUID systems. Both can be achieved on the basis of an improved noise cancellation method.

The existence of much stronger, natural and human-generated external signals results in extremely low (less than one in a million) signal-to-noise-ratio values. These signals are unusable unless methods of external signal (noise) suppression are employed.

The most effective, but also the most expensive and inflexible method of noise suppression is the operation of the method in high-quality magnetically shielded rooms. However, such rooms have been proven to be unacceptable in cardiological practice. Operating outside magnetic shielding and without highly balanced SQUID gradiometer systems is essential for a clinical acceptance. Therefore, the emphasis of recent efforts has been on the development of MCG systems that can be operated in the absence of magnetically shielded rooms. The main technique of noise suppression utilized has been higher order gradiometry.

In diagnostic applications, the magnetic field of the heart may be analyzed spatially and/or over time in order to identify complex changes in cardiac electrical activity due to pathological functional or structural changes in the myocardium. These may result from ischemia, myocardial infarction, volume or pressure changes in the cardiac chambers, or arrhythmia.

Magnetocardiographic imaging by arrays of SQUID sensors is increasingly being investigated for use in the diagnosis of ischemia, heart muscle vitality (differentiation between hibernating and necrotic tissue) and in arrhythmia risk analysis. Biomagnetic localization can be used in cardiology in order to identify focal activity in the cardiac conduction system. Specifically, accessory pathways as in the Wolf-Parkinson-White syndrome, the origin of ventricular extra systoles or ventricular tachycardias may be localized non-invasively with a precision of millimeters.

The potential significance of MCG is that it is a totally noninvasive, non-contact diagnostic and functional imaging method, for which very high sensitivities and specificities have been demonstrated in some clinical studies involving several hundreds of cardiac arterial disease patients.

Magnetocardiograms measured outside magnetic shielding suffer from environmental noise superimposed onto the signal of the heart. One can distinguish three types of noise: homogenous noise (e.g. the magnetic field of the earth), stochastic noise (white noise, colored noise, 1/f noise), and deterministic noise (e.g., power line disturbances with peaks at 50/60 Hz in power spectrum). The homogenous and deterministic noise components often exceed the signal by orders of magnitude. Additionally, stochastic and deterministic noise varies in time so that an adaptive noise cancellation is required.

Deterministic noise components may be either low, medium or high frequency. Low frequency deterministic noise (0.1 to 1 Hz) is typically due to moving elevators, metal doors, metal chairs or other moving metallic (magnetic) objects. Magnetic implants such as defibrillator, pacemakers, sternal wires or dental work may oscillate with the breathing frequency of the patient. Breathing causes a movement of the magnetic parts, which results in an offset in the cardiac time series of usually high amplitude. Moreover, magnetic parts within the body may vibrate due to the mechanical pumping of the heart. The vibration frequency is then strongly correlated to the heartbeat, leading to what is commonly referred to as “correlated noise”.

Middle frequency deterministic noise (1 Hz to 20 Hz) is typically caused by spinning fans, air conditioners, or other clinical apparatus. Vibrations of the building and the system itself as well as flux jumps may also cause disturbances in this middle frequency range.

High frequency noise (>20 Hz) is mostly due to power supplies, monitor frequencies, or other electronic devices.

These various deterministic noise sources make it difficult to extract the useful, undistorted magnetocardiograph [MCG] data that is required for magnetocardiograph analysis.

Many techniques have been exploited in attempts to diminish or remove such unwanted noise from a signal. The most common noise reduction methods utilized have been hardware and software gradiometry techniques combined with classical filtering using low-pass, high-pass or notch filters.

Classical filters of various types have not performed well in this area. Filters are non-adaptive, and their use results in insufficient signal preservation, especially in the case of notch filters.

First or higher order hardware gradiometers have been utilized to provide a suppression of homogenous or gradient fields of lower orders. This method efficiently reduces the influence of the homogeneous magnetic field of the earth, e.g., and has only a small effect on the hearts' signal. However, deterministic and stochastic noise components originating from nearby sources, and having significant spatial gradients are not suppressed sufficiently even by high-precision higher-order gradiometers, which, in addition, are difficult to fabricate and thus expensive.

The most successful prior art method to eliminate deterministic noise is the use of multiple reference sensors. By adaptively applying cross-correlation techniques in various ways (Robinson, ‘Environmental Noise Cancellation For Biomagnetic Measurements’ (1989), and Rueders et al., ‘Frequency Dependent Gradiometry: A New Non-Invasive Method Of Improved Noise Cancellation Applied To Magnetocardiography’ (1989)), it is possible to subtract noise peaks from the signal sensor, provided the noise peaks are correlated. In this context, correlated means that the (deterministic) noise is self-correlated, whereas it is not correlated with the signal.

The problem with the multi-sensor technique is that, for a sufficient noise gradient suppression, at least seven, and up to twenty-five reference sensors are needed.

Furthermore, multiple reference sensors, even when coupled with cross-correlation signal processing, fail to solve a significant problem in signal identification and analysis, that of stochastic noise. Stochastic noise survives the multiple reference sensor procedure since it doesn't correlate at all.

Many attempts have been made to minimize or remove stochastic noise from signals. A method using local projections in state space and the covariance matrix (as in the paper by Schreiber et al. entitled, ‘Nonlinear Noise Reduction For Electrocardiograms’, Chaos 6:87, (1995)) has been shown to be useful in reducing stochastic noise. In this procedure, the signals' signature is localized in state space and is projected onto a noise-free subspace indicated by the largest eigenvalues of the covariance matrix. This method works well, but only if the dimension of the signals' subspace in state space is known.

Generally, in case of high noise levels, the dimension of the signals' subspace in state space is not known and the spectrum of the eigenvalues is flat.

In magnetoencephalography (MEG), mathematical approaches to spatial filtering such as nonlinear beamformers, and specifically synthetic aperture magnetometry, have been used to localize electric and magnetic activity sources in the brain as described in S. E. Robinson and J. Vrba, Comparison of SAM and MUSIC Performance for Unaveraged MEG, and J. Vrba and S. E. Robinson, Differences between Synthetic Aperture Magnetometry and Linear Beamformers, Proceedings of Biomag 2000, 12th International Conference on Biomagnetism, HUT, Espoo, Finland.

Such methods are also helpful for noise separation. However, according to the authors, it is unlikely that synthetic aperture magnetometry or analogous methods could be easily applied to MCG. The main reason is that the human heart represents, at least in the QRS and ST intervals of the cardiac cycle, a spatially extended electric and magnetic source, as opposed to the very local activity sources in the brain.

Hence, until now, no satisfactory technique has been available to substantially reduce all types of noise in magnetocardiograph data.

Some prior art patents and literature in this field are described below. Several patents utilize wavelet transforms to remove noise from a signal.

Abdel-Malek et al., U.S. Pat. No. 5,497,777, entitled Speckle Noise Filtering In Ultrasound Imaging and assigned to General Electric Company, discloses a method of filtering noise from a signal of interest using wavelet transforms. Some of wavelet transform coefficients contributed by the noise components are eliminated, and only the coefficients belonging to the true signal are inverse transformed. The inverse transform recovers an approximation of the true signal without the noise component.

However, this approach is based on the assumption that noise and signal are represented by different coefficients and especially are not overlapping in some coefficients. Additionally, knowledge of which coefficients contain signal information and which contain noise is required in order to reject only those belonging to the noise. Both assumptions are not fulfilled in magnetocardiographic time series.

Kumar et al., U.S. Pat. No. 6,208,951, entitled Method And An Apparatus For The Identification And/Or Separation Of Complex Composite Signals Into Its Deterministic And Noisy Components and assigned to the Council of Scientific & Industrial Research, also discloses a method for separating noise components from a signal of interest using a wavelet transform. A composite signal is wavelet transformed before the noise components are eliminated utilizing the properties of the wavelet transform and its different dimensions to separate the true and noise signals and recover the desired signal.

The problem with this approach is that it requires that signal and noise be separated prior to performing the wavelet transform. This is not the case in measured MCG time series. Therefore, a technique is needed which does not require the prior separation of noise and signal in order to perform the wavelet transform. What is needed is a technique which reorganizes the time series in a way that applying the wavelet transform leads to the desired separation (a steep eigenspectrum).

Tran et al. U.S. Pat. No. 6,249,749, entitled Method And Apparatus For Separation Of Impulsive And Non-Impulsive Components In A Signal and assigned to Ford Global Technologies, Inc., discloses a method for separating two signals within a composite signal by performing a statistical analysis on the wavelet transform coefficients, and detecting their contributions to the different signals. The coefficients contributing to either signal are separately inverse transformed in order to individually recover each signal.

As with Kumar et al., Tran requires the prior separation of signal and noise components. These patents reflect the easiest way to use the wavelet transform for the separation of signal(s) from noise.

Noise reduction techniques are also disclosed in the non-patent literature. L. Rebollo-Neira, A. Costantinides, T. Stathaki, “Signal Representation For Compression And Noise Reduction Through Frame-Based Wavelets”, IEEE Trans. Signal Processing 46(3): 587-597 (1998) discusses a method for noise reduction that uses wavelet transform. This paper mentions suppression of some wavelet subspaces where it is assumed the signal may be noise contaminated.

Leder et al., ‘Reproducibility of HTS-SQUID Magnetocardiography in an Unshielded Clinical Environment’, (International Journal of Cardiology 79 (2-3), July 2001), discloses a technique that measures the magnetic field of the human heart using high temperature superconducting (HTS) sensors. These sensors are operated at the temperature of liquid nitrogen and without electromagnetic shielding. This article highlights the need for a still missing universal noise cancellation technique.

HTS SQUID technology is not yet suitable to measure magnetocardiograms outside shielding. Although there are some promising results, high temperature superconductors are less sensitive compared to low temperature conductors (4-5 times). This will always decrease the system performance such that details in the magnetic signature of the heartbeat won't show up in HTS systems. It is even worse for fetal MCG because the field strength is at least one order of magnitude lower than in adults.

Koch, SQUID magnetocardiography: Status and perspectives, IEEE Transactions On Applied Superconductivity 11: (1) 49-59, Part 1 (March 2001), details recent advances in SQUID-system technology such as improved noise suppression techniques, better field sensitivity (in particular for HTSQUIDs), real time options, vector magnetometers and novel signal analysis approaches have appreciably reduced the technical constraints that hindered until recently the implementation of magnetocardiography techniques into practical clinical use. This article summarizes the state of the art in SQUID magnetocardiography.

Zhang et al., Second-order, High-Temperature Superconducting Gradiometer For Magnetocardiography In Unshielded Environment, Applied Physics Letters 76: (7) 906-908 Feb. 14, 2000, discloses a second-order gradiometer for magnetocardiography in unshielded environment. This high-temperature SQUID system is demonstrated to be diagnostically relevant for magnetocardiograph in terms of signal-to-noise ratio, spatial resolution, frequency bandwidth, rejection of environmental disturbances, and long-term stability considerations. Zhang discloses an unshielded single channel system in a transportable Dewar, which can be used directly at the patient's bed. Compared to low temperature superconductor SQUID performance, it is very weak. However, its performance may be sufficient for its narrow intended use for monitoring ST-segments in infarction patients.

Robinson, Environmental Noise Cancellation For Biomagnetic Measurements, Advances in Biomagnetism, Plenum Press, New York 1989, provides a general description of the state of the art in biomagnetic denoising. This article describes the use of reference sensors and noise cancellation based on cross-correlation techniques.

This article is the principal authority for cross correlation denoising. The approach described in the article is presently being utilized commercially for the denoising. In this article, the minimum number of needed reference sensors was found to be 7.

Denoising By Soft-Thresholding, IEEE Trans. Inform. Theory 41:613, (1995) discloses that the hard or soft threshold of wavelet coefficients is well suited for signal recovery even in state space as described by Effem et al. “Nonlinear Denoising Of Transient Signals With Application To Event Related Potentials”, Physica D 140(3-4), Jun 15, (2000).

This article proposes removing transients from an EEG time series. Event related potentials (ERPs) are evoked by applying a stimulus to a patient. A corresponding region in the brain shows a particular responding waveform that is, according to its polarity and time-after-event classified. Effern analyzed the P300 which is a very weak wave with a signal-to-noise-ratio (SNR) of much below 1. Since the P300 usually occurs only for some milliseconds a denoising is very difficult.

Effern's key concept is so called circular embedding. He used Takens' theorem to embed an artificial time series that he created by continuously adding all single P300 time series leading to one “big” time series. Wavelet transforming of embedded vectors helped him to identify transients, which he then removed.

Whatever the precise merits, features and advantages of the above mentioned prior art, none of them disclose a common technique to substantially reduce all types of noise in magnetocardiograph data.

It addition to its other uses, it would be highly desirable to develop such a procedure for use in fetal magnetocardiography. Fetal magnetocardiography has potential as an alternative method of fetal surveillance. Since fetal heart signals are 10 times weaker than those of adults, a better magnetic field resolution is required (<10 fT/Hz^{1/2} versus<50 fT/Hz^{1/2} for adults). Fortunately, a rather limited signal bandwidth of 25 Hz is usually sufficient.

Thus far, only fetal magnetocardiography inside magnetically shielded rooms (MSR) has been convincingly demonstrated and reported in the literature. Attempts to use gradiometers without shielding, especially HTS gradiometers, have been, thus far, relatively unsuccessful. In the third trimester of pregnancy, it is not reliably possible to measure electrical activity using abdominal leads. This is due to the presence of an electrically insulating layer, vernix caseosa, on the fetus during this period. As magnetic fields propagate relatively undisturbed through body tissue, it is possible to record the fetal magnetocardiography more precisely than the fetal ECG.

Fetal magnetocardiography may be used to examine signal morphology, cardiac time intervals and heart rate variability. This will allow the assessment of the fetal cardiac conduction system, arrhythmias, cardiac congenital defects, growth, development of the autonomic nervous system, acidosis and fetal stress.

An overview of the current status of fetal heart diagnostics based on fetal magnetocardiography is given in Fetal Biomagnetism in Frontiers in Fetal Health 1:(5) November 1999, Satellite Symposium of the 4^{th} Hans Berger Conference, Jena, Germany, Sep. 26, 1999, Ed., A. L. Pastuszak.

The significance of fetal magnetocardiography resides in its unique monitoring and diagnostic capabilities. The various reported and possible diagnostic uses of fetal magnetocardiography can be broken down in two periods of application: during gestation and at the time of delivery.

During gestation fetal magnetocardiography may be used in 1] the analysis of cardiac rhythm, especially when a cardiac arrhythmia or a conduction disturbance (AV block) is suspected; 2] the analysis of the PR interval in the fetus and diagnosis of 1st degree AV block in the fetal population at risk (Lupus Erythematosus, autoimmune disease, etc.); 3] the analysis of the amplitude of the QRS complex and diagnosis and follow up of the fetus with ventricular hypertrophy (fetus of diabetic mother, mother receiving steroids, etc.); 4] the analysis of repolarization phase (e.g., ST segment changes related to fetal ischemia); 5] assessment of the fetus well being (heart rate variability); and 6] the detection of fetus at risk from long QT syndrome for which fetal magnetocardiography may be the only method available.

During the intrapartum period fetal magnetocardiography may be used in 1] assessment of the fetal well being during the different phases of delivery (HRV study); 2] direct analysis of the AV conduction (PR interval) to provide useful information on the fetal well being/ distress; and 3] ST segment analysis to provide useful information on cardiac ischemia during fetal distress.

It is therefore an object of this invention to provide a procedure for detecting and analyzing fetal health during gestation and intrapartum periods.

It is also an object of this invention to provide an effective system and method to substantially eliminate deterministic and stochastic noise from measured magnetocardiograph or electrocardiograph time series.

It is an object of this invention to provide adaptive noise cancellation methods, particularly with reference to de-noising signals obtained from magnetocardiography or electrocardiography.

It is another object of this invention to provide adaptive noise cancellation methods utilizing only one reference sensor to remove stochastic noise.

It is an object of this invention to provide adaptive noise cancellation methods where the signals subspace in state space is not known.

It is another object of this invention to provide adaptive noise cancellation methods where a simple wavelet transform of the time series has a flat eigenspectrum, which would ordinarily preclude separation of the signal from noise components.

### SUMMARY OF THE INVENTION

The present invention provides for a system and method to substantially eliminate deterministic and stochastic noise from measured magnetocardiograph or electrocardiograph time series more effectively than known prior art methods. It requires only that the signal be approximately deterministic. This is the case when magnetocardiograph or electrocardiograph time segments of four seconds or longer duration are used.

### DESCRIPTION OF THE DRAWINGS

FIG. 1a represents an observed system viewed in terms of time.

FIG. 1b represents an observed system viewed in terms of reconstructed state space and shows the densely lying trajectories of an at least approximately deterministic system.

FIG. 1c depicts a portion of the state space of FIG. 1b before and after the introduction of noise to the signal.

FIG. 1d is a multi-resolution representation of the state space vectors in the wavelet domain.

FIG. 1e illustrates the high entries in wavelet coefficients representing signal related directions and low entries for those of stochastic noise related directions.

FIG. 2a represents 5 seconds of electrocardiograph data recorded at 200 Hz as the pure signal recorded by the main sensor.

FIG. 2b shows the frequency spectrum of the electrocardiograph after pre-filtering by a 50 Hz notch filter and a second-order low pass filter at 100 Hz.

FIG. 2c shows the signal of FIG. 2b with added white noise superimposed.

FIG. 2d shows the resulting noise spectrum of FIG. 2c.

FIG. 2e shows the cleaned time series, after wavelet transformations and subtraction in state space of the signal of FIG. 2c.

FIG. 2f shows the frequency spectrum of the electrocardiograph after wavelet transformations and subtraction in state space.

FIG. 3a represents 5 seconds of magnetocardiograph signal recorded outside a shielding room where only the main component of the heart signal (R wave) is visible.

FIG. 3b represents the frequency spectrum of the signal of FIG. 3a.

FIG. 3c represents 5 seconds of the simultaneously recorded noise signal of FIG. 3a.

FIG. 3d represents the Fourier spectrum of the signal shown in FIG. 3c.

FIG. 3e shows the time series resulting from the present de-noising procedure.

FIG. 3f shows the Fourier spectrum corresponding to the FIG. 3e time series.

FIG. 4a shows the original time series using the data of Example 1.

FIG. 4b represents the frequency spectrum of the signal of FIG. 4a.

FIG. 4c shows the time series after noise reduction with ghkss.

FIG. 4d represents the power spectrum of the signal of FIG. 4c.

FIG. 4e depicts the residuum of noise in the signal of FIG. 4a using the present de-noising method.

FIG. 4f depicts the residuum of noise in the signal of FIG. 4a after noise reduction with ‘ghkss’.

FIG. 5a depicts an excerpt of three seconds of a time series recorded from a pregnant woman with a low temperature SQUID within shielding.

FIG. 5b shows some of the typical noise peaks at 50 Hz are missing, which indicates the use of a shielding chamber.

FIG. 5c depicts the result after applying NLD showing the MCG of the mother visible but contaminated with low frequent (respiratory) artefacts, which may be removed by increasing the observation time.

FIG. 5d depicts the power spectrum, free from noise peaks and showing a decreased white noise level.

FIG. 5e depicts the spectrum of the QRS complexes of the foetal MCG after removal of the mother's MCG from the time series and applying NLD again, demonstrating that previously overlapping heartbeats have been separated.

FIG. 5a depicts the spectral energy of the mother's MCG.

FIG. 5f depicts the spectral energy of the foetal MCG, which is much lower but lies within the same bandwidth as that of the mother (d) and demonstrates the importance of highly adaptive denoising procedures.

### DESCRIPTION OF THE PREFERRED EMBODIMENTS

I have discovered the hard or soft threshold of wavelet coefficients is well suited for signal recovery in state space and have applied this technique to the de-noising of magnetocardiograph or electrocardiograph time series signals.

The present invention provides a method and system for nonlinear de-noising (NLD) of magnetocardiograph or electrocardiograph time series signals by performing local projections in the reconstructed state space using the wavelet transform to identify and describe deterministic structures. Thus, the goal is to locate and separate subspaces generated by any deterministic process independent of its source (be it the noise or the signal of the heart). The method consists of first separating a subspace from stochastic noise followed by separating different subspaces.

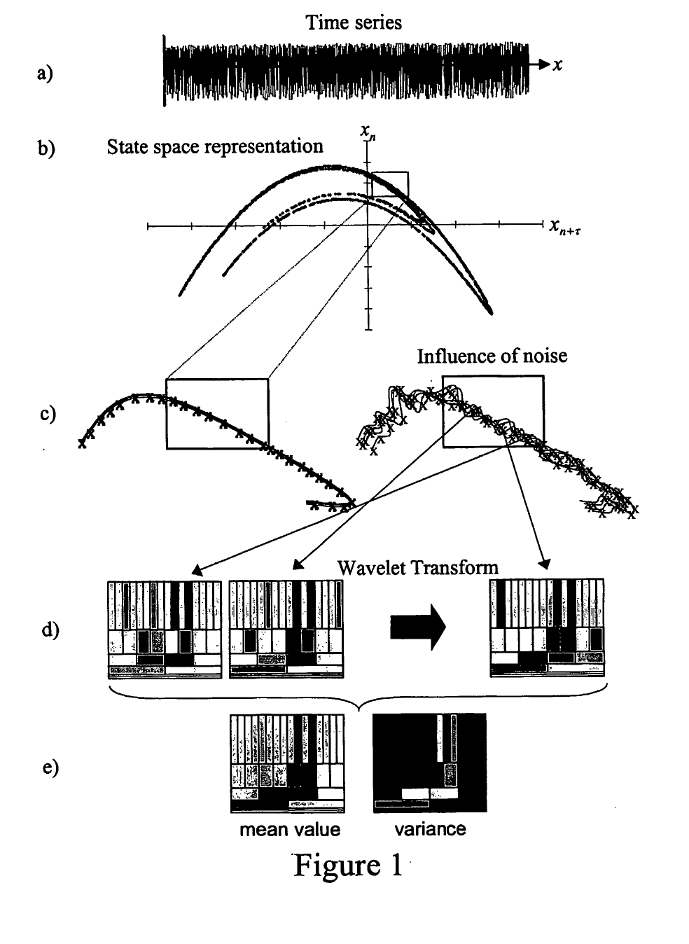

To represent the dynamical properties of an observed system it is useful to operate in the reconstructed state space (see F. Takens, ‘Detecting Strange Attractors in Turbulence’, Lecture notes in math., Springer, N.Y., 1981) instead of the time domain (FIG. 1a). An at least approximately deterministic system leads to densely lying trajectories and is constrained to a subspace (FIG. 1b) whereas a stochastic process causes a random distribution within the entire state space. Superimposing white noise onto a deterministic signal causes distortions of the primary densely lying trajectories (FIG. 1c).

To identify and to describe a deterministic structure in state space, it is useful to transform the state space vectors into a suitable basis system. “Suitable” means that one attempts to find a basis function, which adapts best to the specific deterministic structure present.

It is possible to describe the determinism by only a few coefficients in the domain of the new basis system, due to the fact that directional information is compressible. In contrast, stochastic noise is incompressible and, therefore, needs a complete set of basis coefficients to be reproduced.

The wavelet transform provides many highly adaptive basis functions called wavelets.

It is defined by translations and dilations of a basis function (a wavelet) convolved with a signal x(t). An additional scaling factor (mostly a power of 2) lets the wavelet transform act like a mathematical microscope; it lets one observe signal information at different scales dependent on its location. Exactly this property is useful, because one obtains a multi-resolution representation of the state space vectors in the wavelet domain (FIG. 1d).

The general mathematical background of wavelet transforms is well known and an introduction can be found in the paper by Mallat et al. entitled A Theory For Multiresolution Signal Decomposition: The Wavelet Representation, IEEE Trans. Pat. Rec. Mach. Intel. 11:674, (1989). A comprehensive database containing the available literature and wavelet applications is presented in Amara Graps website:

http://www.amara.com/current/wavelet.html.

It is important to choose an optimum wavelet. An optimally chosen wavelet (analyzing function) is one which best represents the signal. For example, in fast Fourier transform, the analyzing functions are sine and cosine waves. Applied to a pure sine wave, the fast Fourier transform yields a single peak in the spectrum. However, applying a fast Fourier transform to a rectangular pattern requires huge amount coefficients to properly describe this pattern. The same is true with the wavelets: the better the wavelet matches the function-of-interest (here: heartbeat) the better. It is possible to design a problem-oriented wavelet, one of the big advantages of the wavelet transform. For the purposes of this invention, the best choice in this case is the well-known Coiflet using filterorder 6. Other Coiflet wavelet transforms may be used, as well as Haar, Morlet, Mexican Hat, biorthogonal spline, Daubechies, Malvar, Lemarie, Meyer, and Symlet wavelet types.

The optimally chosen wavelet provides high entries in wavelet coefficients representing signal related directions and low entries for those of stochastic noise related directions (FIG. 1e). This allows the definition of a shrinking condition for the projection towards the direction of the maximal variance effectuated by the determinism of the signal. Finally, the inverse wavelet transform recovers the state space vectors from which the cleaned time series can be reconstructed.

The deterministic noise fills additional subspaces, which have to be separated from the manifold of the signal. The noise related subspaces are localized and described by recording the noise in an additional reference sensor and transforming the state space vectors into the wavelet basis system. Then, their signature in the time series of the source sensor is identified and a simple subtraction in state space is performed. This procedure is superior to common cross-correlation techniques because the dynamical properties of the deterministic noise are considered. It is believed that the wavelet transform has never been used for this purpose, especially not in conjunction with reference sensors.

The noise reduction methods described are particularly useful in obtaining useful data from magnetocardiographs. One particularly beneficial use of the cleaned signal is in determining the well being of a fetus carried by a pregnant mammal, especially a human being. During certain phases of pregnancy the fetal ECG is very difficult to record because of the insulating fat layer in the fetus. Since the magnetic permeability of tissue is that of free space, MCG's of the fetus do not suffer from this failing. However, until now, it has been impossible to diagnose the presence of cardiac abnormalities in the fetus using SQUID systems outside shielding due to the very weak signal of the fetus, and an unusable low signal-to-noise-ratio. Using the techniques described herein it is now possible to separate the signals received from the mother from those of the fetus and to determine abnormalities in the fetal heartbeat.

The disclosed NLD technique also provides significant advantages in conjunction with SQUID technology. A shielded room is not necessary in SQUID magnetocardiography, however the absence of shielding results in increased noise and requires more powerful noise cancellation techniques such as that described herein.

One of the key aspects of the inventive method is the use of adaptive thresholding. As used herein, thresholding means dividing the eigenspectrum of the wavelet coefficients.

After embedding the time series into the state space, nearest neighbor search is performed for each single state space vector “x” and the wavelet transform is applied. Then, a center-of-mass wavelet is created by building the mean from all transformed vectors that are the nearest neighbors to “x”.

If it were possible to perfectly separate subspaces occupied by noise and signal a hard thresholding could be performed. In that case all coefficients belonging to noise are set to zero and the rest are kept as it is. However, since, in general, subspaces overlap, an adaptive thresholding is required, which accommodates the fact that some coefficients contain both signal and noise information.

In soft thresholding, noise coefficients are set not to zero (hard) but to a certain value, e.g. the mean value (soft). This keeps some information of these particular coefficients but decreases their importance. The more noise that overlaps with the subspace of the signal the more difficult it is to separate them and the more important adaptive thresholding becomes

The concept underlying the mathematical methodology of NLD is the performance of local projections in the reconstructed state space using the wavelet transform to identify and describe deterministic signal structures. The goal is to locate and separate subspaces generated by any deterministic process independent of its source (be it the noise or the signal of the heart). The procedure consists of two parts: (1) the separation of a subspace from stochastic noise and (2) the separation of different subspaces, which are described below.

To represent the dynamical properties of an observed system it is useful to operate in the reconstructed state space instead of the time domain. FIG. 1a shows the time domain plot of the x-component of a sample time series, which is known as Hénon map and defined as follows:

xₙ₊₁=1.4−xₙ ²+0.3yₙ

yₙ₊₁=xₙ

Obviously, it is impossible to recognize any dynamical property of the underlying (deterministic) system. Time delay embedding of the Hénon map leads to the following state space vectors:

[symbol]

(n)=(xₙ, xₙ₋ᵣ, xₙ₋₂ᵣ, . . . , xₙ₋₍ₘ₋₁₎ᵣ)

where τ denotes the time delay and m the embedding dimension. The state space representation of the Hénon map is given in FIG. 1b). Here, using τ=1 and m=2 the components of the state space vectors are depicted in a two dimensional graph by plotting component xₙ₊ᵣ against xₙ. An at least approximately deterministic system leads to densely lying trajectories and is constrained to a subspace whereas a stochastic process causes a random distribution within the entire state space.

Superimposing white noise to a deterministic signal causes distortions of the primary densely lying trajectories. The left graph of FIG. 1c shows an excerpt of some (bunched) trajectories of FIG. 1b. The effect of superimposing noise to this excerpt is demonstrated in the right part of FIG. 1c.

The next step is to identify and to describe a deterministic structure in state space. For this purpose it is useful to transform the state space vectors into a suitable basis system. “Suitable” means that one attempts to find a basis function that adapts best to the deterministic structure. In this case it is possible to describe the determinism by only a few coefficients in the domain of the new basis system. This is due to the fact that directional information is compressible. In contrast, stochastic noise is incompressible and, therefore, would need a complete set of basis coefficients to be reproduced.

The wavelet transform provides many highly adaptive basis functions called wavelets. It is defined by translations and dilations of a basis function (a wavelet) convolved with a signal x(t). An additional scaling factor (mostly a power of 2) lets the wavelet transform act like a mathematical microscope, which means that it lets one observe signal information at different scales dependent on its location. Exactly this property is useful, because one obtains a multi-resolution representation of the state space vectors in the wavelet domain (see FIG. 1d).

With an optimally chosen wavelet one can expect high entries in wavelet coefficients representing signal related directions and low entries for those of stochastic noise related directions (FIG. 1e). This enables one to define a shrinking condition for the projection towards the direction of the maximal variance effectuated by the determinism of the signal. Finally, the inverse wavelet transform recovers the state space vectors from which the cleaned time series can be reconstructed.

Adaptive (hard or soft) thresholding of wavelet coefficients is well suited for signal recovery even in state space and is important in de-noising of MCG or ECG time series signals.

The deterministic noise fills additional subspaces, which have to be separated from the manifold of the signal. In application to MCG, the noise related subspaces are localized and described by recording the noise in an additional reference sensor and transforming the state space vectors into the wavelet basis system. Then, their signature in the time series of the source sensor is identified and a simple subtraction in state space is performed. This procedure is superior to common cross-correlation techniques because the dynamical properties of the deterministic noise are considered.

The significance of NLD resides in its potential ability to separate weak useful bioelectric or biomagnetic signals from many orders of magnitude stronger noise, without recurring to intensive signal averaging and filtering (both of which distort the signal to be measured.)

To demonstrate the efficiency of the novel de-noising scheme, it was applied to simulated signals using electrocardiographic data of a healthy patient wherein the data is recorded at 200 Hz as the pure signal recorded by a main sensor

### EXAMPLE 1

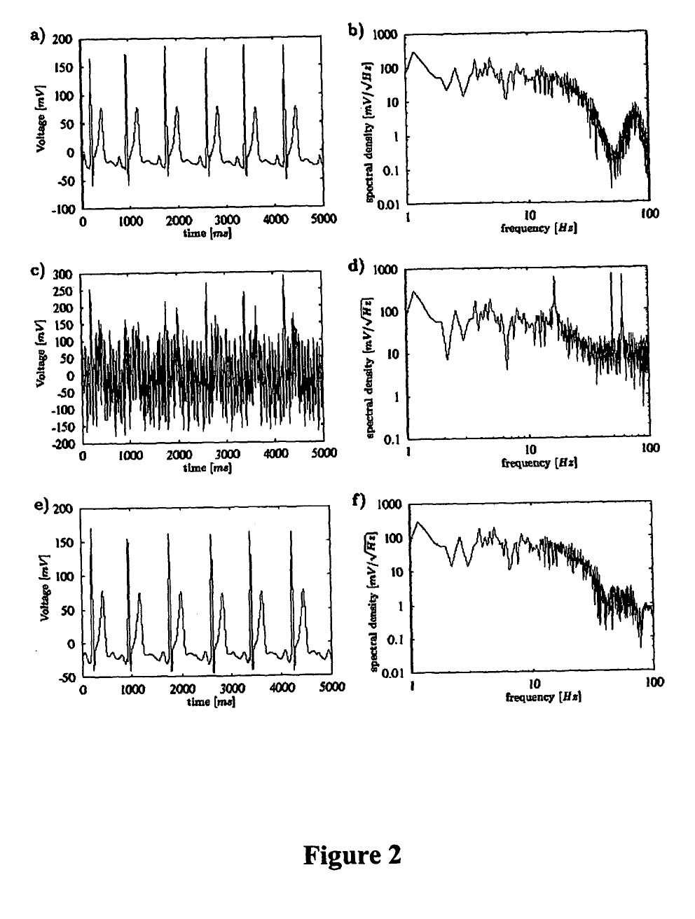

NLD was applied to simulated noisy signals, starting from a 5 second ECG recording of a healthy heart, recorded at 200 Hz bandwidth, and taken as the pure signal from the main sensor. This ECG was pre-filtered by a 50 Hz notch filter and a second-order low pass filter at 100 Hz (FIGS. 2a and 2b).

Subsequently, white noise is added with an amplitude variance of 30% referred to the electrocardiograph's variance, and the deterministic noise. The deterministic noise had frequency peaks at 16⅔ Hz, 50 Hz (rail power supply in Europe and subharmonics), and 60 Hz (signal analysis systems) with an amplitude variance of 100%.

The deterministic noise had frequency peaks at 16⅔ Hz, 50 Hz (power supply in Europe and subharmonics), and 60 Hz (signal analysis systems) with an amplitude variance of 100% (see FIGS. 2c and 2d). A reference noise time series was created using the same parameters as mentioned above, but additionally, with variations in amplitude and a constant phase shift for the deterministic noise components.

FIG. 2c shows the signal with added white noise superimposed, and FIG. 2d the resulting noise spectrum. The reference time series is generated by creating noise using the same parameters as mentioned above, but additionally, with variations in amplitude and a constant phase shift for the deterministic noise components.

After wavelet transformations and subtraction in state space, FIG. 2e shows the cleaned time series. A reference time series is generated by creating noise using the same parameters as mentioned above, but additionally, with variations in amplitude and a constant phase shift for the deterministic noise components. FIG. 2f shows the frequency spectrum of the electrocardiograph after wavelet transformations and subtraction in state space.

One can infer from FIG. 2e that the baseline between the heartbeats (a good indicator of the de-noising quality) is almost noise free. Hence, the present invention's method performed both signal preservation and considerable noise reduction.

### EXAMPLE 2

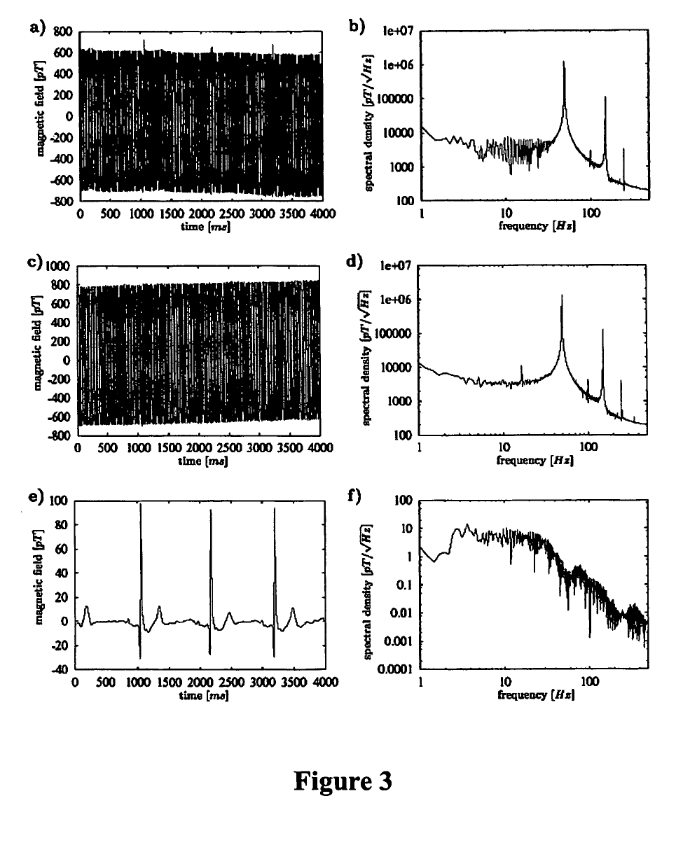

As an example of measured signal data, data obtained from the magnetocardiograph of a healthy patient recorded outside a shielding room using a laboratory HTSQUID system is depicted in FIG. 3. Five [5] seconds of magnetocardiograph signal was obtained as depicted in FIG. 3a. The patient's heartbeat is only barely visible in FIG. 3a. A simultaneously recorded noise time series was recorded as depicted in FIG. 3c.

The frequency spectrum of the signal depicted in FIG. 3a is shown in FIG. 3b; that depicted in FIG. 3c is shown in FIG. 3d. Due to the width of the 50 Hz peak in the spectrum no notch filter was used.

For this measurement, two axial gradiometers of first order with 7 cm baseline were mounted at a distance of 7 cm one above the other. In this example, the top gradiometer recorded the reference signal (FIGS. 3c and 3d).

FIGS. 3e and 3f show the time series along with its corresponding Fourier spectrum resulting from the present de-noising procedure. In the reconstructed magnetocardiograph [MCG] of FIG. 3e, even small details of the heartbeat are revealed. Again, the baseline between the heartbeats is almost noise free.

### EXAMPLE 3

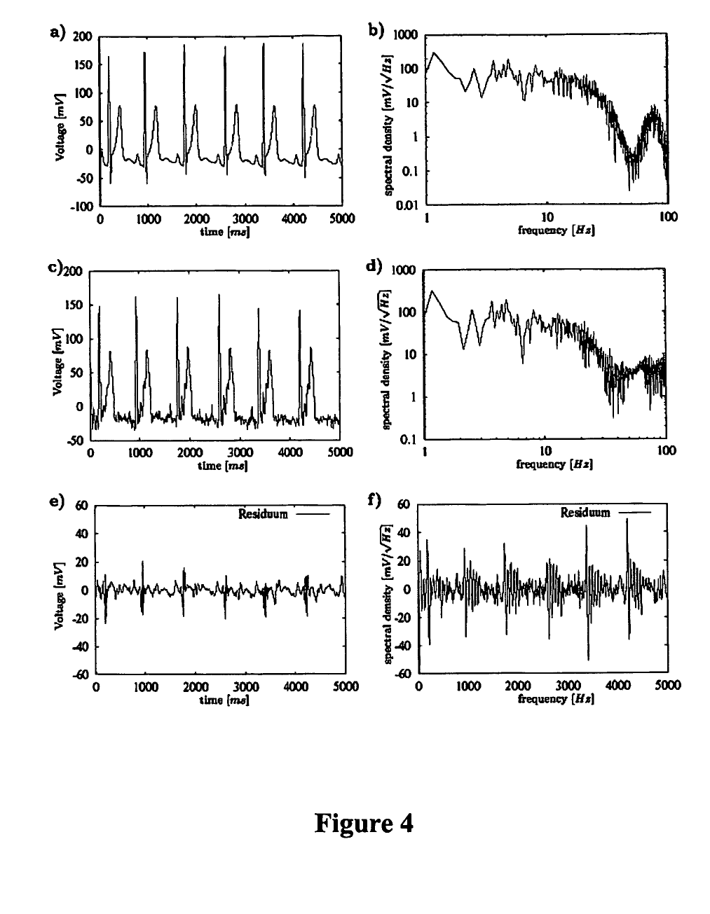

FIG. 4a-b illustrates the superiority of the present invention's system and method over one of the prior art de-noising techniques.

The analysis of the Example 2 data set based upon this method is shown in FIGS. 4a and 4b. The tool ‘ghkss’ described in the paper by Hegger et al. entitled, ‘Nonlinear Time Series Analysis (TISEAN)’, incorporated herein by reference, is used to analyze the data set. This is the algorithmic form of “Nonlinear Noise Reduction For Electrocardiograms” (Chaos 6:87,1995).

The tool ‘ghkss’ was applied to the data set and obtained the results shown in FIGS. 4c and 4d. Obviously, NLD reaches a better noise reduction quality in this case, clarified by the respective residuums (see FIGS. 4e and 4f). This is due to the fact that ‘ghkss’ is not able to separate overlapping subspaces in state space, which is one of the most important features of NLD.

To illustrate this, an analysis of the same data is performed based upon the technique described in the paper by Schreiber et al. entitled, ‘Nonlinear Noise Reduction For Electrocardiograms’ (Chaos 6:87, 1995), the disclosure of which is incorporated herein by reference. In summary form, the procedure reduces stochastic noise by performing local projections in state space using the covariance matrix. The signals' signature is localized in state space and is projected onto a noise-free subspace indicated by the largest eigenvalues of the covariance matrix. This method works well, but only if the dimension of the signals' subspace in state space is known.

The results of the analysis are shown in FIGS. 4c and 4d. It should be noted that NLD reaches a better noise reduction quality in this case. This is also demonstrated by the respective residuums of noise (FIGS. 4e and 4f). The NLD residuum is much lower than that of ‘ghkss’. This is due to the fact that, in contrast to NLD, ‘ghkss’ is not able to separate overlapping subspaces in state space, while NDL does. That separation ability is one of the most important features of NLD.

NLD was also compared with another existing technique, Frequency Dependent Gradiometry (FDG) and NLD were applied to the same MCG sample, and it turned out that NLD performed a much superior noise reduction.

### EXAMPLE 4

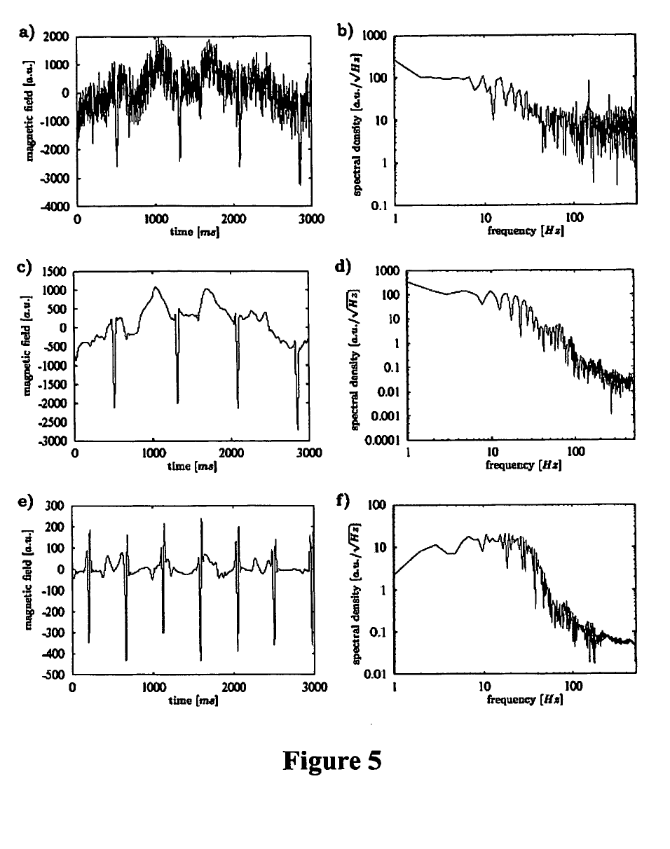

The example shows the applicability of the inventive method to measurement of a foetal heartbeat using MCG. FIG. 5a shows an excerpt of three seconds of a time series recorded from a pregnant woman with an LTSQUID within shielding. In FIG. 5b some of the typical noise peaks at 50 Hz are missing, which indicates the use of a shielding chamber. In the first NLD step the deterministic noise components are removed.

FIG. 5c shows the result after applying the second NLD step. The MCG of the mother is visible being still contaminated with low frequent (respiratory) artefacts, which may be removed by increasing the observation time. Its power spectrum in FIG. 5d is free from noise peaks and shows a decreased white noise level.

Removal of the mother's MCG from the time series and applying NLD again, the QRS complexes of the foetal MCG are obtained as shown in FIG. 5e. Note that even previously overlapping heartbeats are separated. The spectral energy of the foetal MCG shown in FIG. 5f is much lower but lies within the same bandwidth as that of the mother's shown in FIG. 5d. This further demonstrates the importance of highly adaptive denoising procedures.

The programming of the present invention may be implemented by one of skill in the art of digital signal processing.

The above examples demonstrate the effective implementation of a nonlinear noise reduction method for magnetocardiograms using wavelet transforms. While various preferred embodiments have been shown and described, it will be understood that there is no intent to limit the invention by such disclosure, but rather, it is intended to cover all modifications and alternate constructions falling within the spirit and scope of the invention, as defined in the appended claims.

1. A method for nonlinear de-noising of magnetocardiograph or electrocardiograph time series signals comprising performing local projections in the reconstructed state space using the wavelet transform to identify and describe deterministic structures.

2. A method for nonlinear de-noising of magnetocardiograph or electrocardiograph time series signals comprising applying the wavelet transform to identify and describe magnetocardiograph or electrocardiograph related subspaces in state space.

3. A method for removing deterministic noise from the manifold of the signal from a magnetocardiograph or electrocardiograph comprising:
recording the signal in a main signal sensor and at least one reference sensor separate from the main signal sensor;
reconstructing the state space operating on the measured time series using Takens theorem;
transforming the state space vectors into a wavelet basis system;
identifying the signature of the state space vectors of the noise in the time series of the reference sensor and relocate this signature in the time series of the source (signal) sensor;
subtracting the state space vectors of the noise in state space; and
reconstructing the cleaned magnetocardiograph signal.

4. A method as claimed in claim 1 wherein the source of the signal is from suitable sensors appropriately located in the magnetocardiograph or electrocardiograph apparatus.

5. A method as claimed in claim 1 wherein the step of wavelet identification and/or separation of composite signals is used for discrete, biorthogonal, and continuous wavelets.

6. A method as claimed in claim 1, wherein the wavelet type used is selected from the group consisting of Haar, Morlet, Mexican Hat, biorthogonal spline, Daubechies, Malvar, Lemarie, Coiflet, Meyer, and Symlet wavelet types.

7. A method as claimed in claim 6, wherein the wavelet type used is a Coiflet wavelet type.

8. A method as claimed in claim 6, wherein the wavelet type used is a Coiflet filterorder 6 wavelet type.

9. A method as claimed in claim 1 wherein the identification and/or separation of composite signals is done by dividing the signal into a number of sub-interval signals and applying recursive wavelet transformation to each subinterval signal.

10. A method for separating a sampled composite signal from a magnetocardiograph or electrocardiograph containing stochastic and deterministic noise into its signal and noise components comprising:
(a) recording the signal in a main signal sensor and in at least one reference sensor separate from the main signal sensor;
(b) separating subspaces from stochastic noise;
(c) separating different subspaces belonging to individual dynamical processes;
(c) transforming the state space vectors into a wavelet basis system;
(d) identifying the signature of the state space vectors of the noise in the time series of the source sensor;
(e) subtracting the reconstructed noise from the signal time series; and
(f) reconstructing the cleaned magnetocardiograph signal.

11. A method as claimed in claim 10 for de-noising the signal received from a SQUID magnetocardiograph apparatus.

12. A method as claimed in claim 1 for de-noising the signal from an unshielded magnetocardiograph.

13. The use of the method of claim 1 to determine the existence of subspaces in state space created by a heart disease selected from the group consisting of arrhythmia, myocardial infarction, and ischemia.

14. The use of the method of claim 1 for 24 hour recording using portable electrocardiographs.

15. The use of the method of claim 1 to separate signals from a magnetocardiograph of mother and foetus.
